(19) United States
(12) Patent Application Publication
(10) Pub. No.: US 2010/0262157 A1
(43) Pub. Date: Oct. 14, 2010

(54) Methods and Systems for Loading a Stent

(75) Inventors: Michele Silver, Windsor, CA (US); Karan Punga, Santa Rosa, CA (US); William Chang, Windsor, CA (US); William Berthiaume, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/423,285

(22) Filed: Apr. 14, 2009

(51) Int. Cl. A61F 11/00; A61F 2/06

(52) U.S. Cl. 606/108; 623/1.11; 623/1.23

(57) ABSTRACT

A stent loading and delivery system includes a delivery catheter having a catheter lumen, a stent loading assembly adjacent a distal end of the delivery catheter, and a braided stent disposed on the stent loading assembly. The stent loading assembly is configured to retain the stent in an expanded state until just prior to insertion into a patient at which time the stent loading assembly elongates and compresses the stent into a delivery configuration.

### TECHNICAL FIELD

The technical field of this disclosure is medical implant devices, particularly, braided stents.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTA (percutaneous transluminal angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents act as a scaffold to support the lumen in an open position. Various configurations of stents include a cylindrical tube defined by a mesh, interconnected stents or like segments. Balloon-expandable stents are mounted on a collapsed balloon at a diameter smaller than when the stents are deployed. Stents can also be self-expanding, growing to a final diameter when deployed from a shaft or like device.

One approach has been to fabricate stents from braided metal or polymer fibers and combinations thereof. Unfortunately, braided stents often undergo stress relaxation in the delivery system prior to deployment, leading to a smaller post-deployment diameter. This may result in a lack of radial strength to prop open the vessel lumen. One approach to alleviate this problem has been to increase the diameter of the fibers forming the braided stent to increase the radial strength. Unfortunately, this increases the crossing profile of the compressed stent, reducing maneuverability and the ability to deploy the stent in smaller vessels. An increased fiber diameter may also increase the time for a bioabsorbable stent to be absorbed and interrupt blood flow dynamics.

It would be desirable to have a braided stent delivery system and method that would overcome the above disadvantages.

### SUMMARY OF THE INVENTION

One aspect of the present invention provides a stent loading and delivery system. The system includes a delivery catheter having a catheter lumen; a stent loading assembly adjacent a distal end of the delivery catheter; and a stent disposed on the stent loading assembly, wherein the stent comprises a braided stent framework having a first framework end and a second framework end.

Another aspect of the present invention provides a method of loading and delivering a braided stent. The method including the steps of providing a braided stent, the braided stent disposed on a stent loading assembly; retracting an inner sheath and an outer sheath; deploying at least one hook from the inner sheath and at least one hook from the outer sheath based on the retraction; grasping a portion of the braided stent with each of the deployed hooks; and elongating and compressing the grasped stent.

Another aspect of the present invention provides a method of loading and delivering a braided stent. The method includes providing a braided stent, the braided stent disposed on a stent loading assembly, inserting the stent loading assembly into a delivery catheter, compressing the braided stent based on the insertion of the stent loading assembly into the delivery catheter; and loading the stent within the delivery catheter based on the compression of the braided stent.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a perspective view of a stent delivery system made in accordance with the present invention.

FIGS. 2 to 5 are side views of a distal portion of a braided stent delivery system made in accordance with the present invention.

FIG. 6 is a detailed portion of a braided stent with engaged hook assemblies made in accordance with the present invention.

FIGS. 7A to 7C are side views of the stent delivery system shown in FIGS. 2 to 5 within a vessel in accordance with the present invention.

FIGS. 8 to 11 are side views of a distal portion of another embodiment of a braided stent delivery system made in accordance with the present invention.

FIGS. 12 to 16 are side views of a distal portion of another embodiment of a braided stent delivery system made in accordance with the present invention.

FIGS. 17 to 21 are side views of a distal portion of another embodiment of a braided stent delivery system made in accordance with the present invention.

FIGS. 22 to 25 are side views of a distal portion of another embodiment of a braided stent delivery system made in accordance with the present invention.

FIGS. 26 to 29 are side views of a distal portion of another embodiment of a braided stent delivery system made in accordance with the present invention.

FIGS. 30 to 32 are side views of a distal portion of another embodiment of a braided stent delivery system made in accordance with the present invention.

FIG. 33 is a side view of a distal portion of another embodiment of a braided stent delivery system made in accordance with the present invention.

FIGS. 34 and 35 are side views of a distal portion of one embodiment of a mechanical hook made in accordance with the present invention.

FIGS. 36 and 37 are side views of a distal portion of another embodiment of a mechanical hook made in accordance with the present invention.

FIGS. 38 and 39 are side views of a distal portion of another embodiment of a braided stent delivery system made in accordance with the present invention.

FIGS. 40 and 41 are end views of the collapsible stopper of the embodiment shown in FIGS. 38 and 39.

FIG. 42 is a flow chart of a method of loading and delivering a braided stent in accordance with the present invention.

### DETAILED DESCRIPTION OF PRESENTLY PREFERRED EMBODIMENTS

The invention will now be described by reference to the figures wherein like numbers refer to like structures. The terms “distal” and “proximal” are used herein with reference to the treating clinician during the use of the catheter system; “Distal” indicates an apparatus portion distant from, or a direction away from the clinician and “proximal” indicates an apparatus portion near to, or a direction towards the clinician.

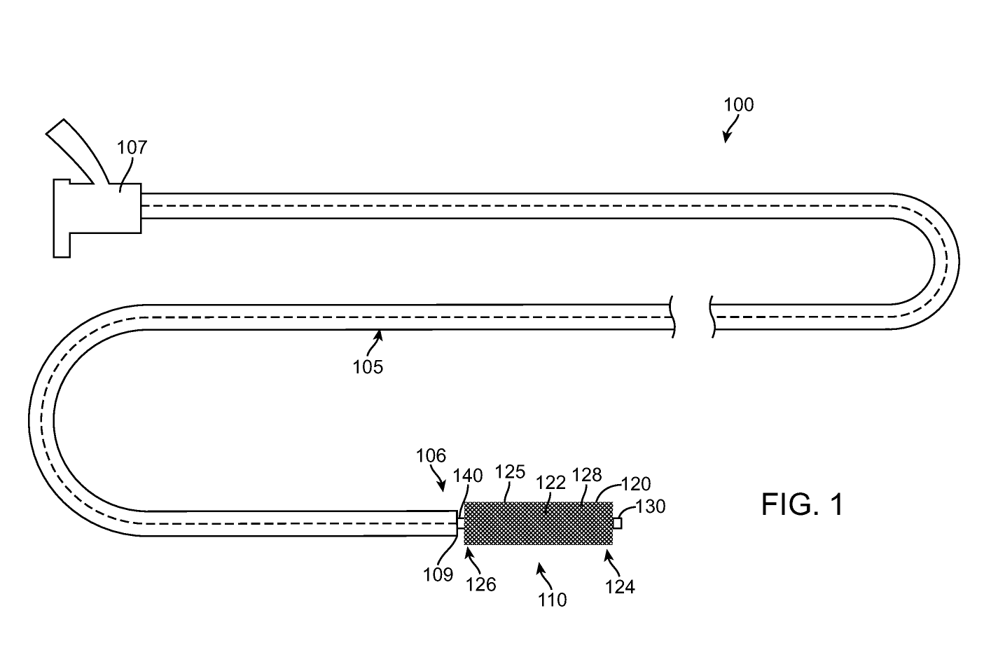

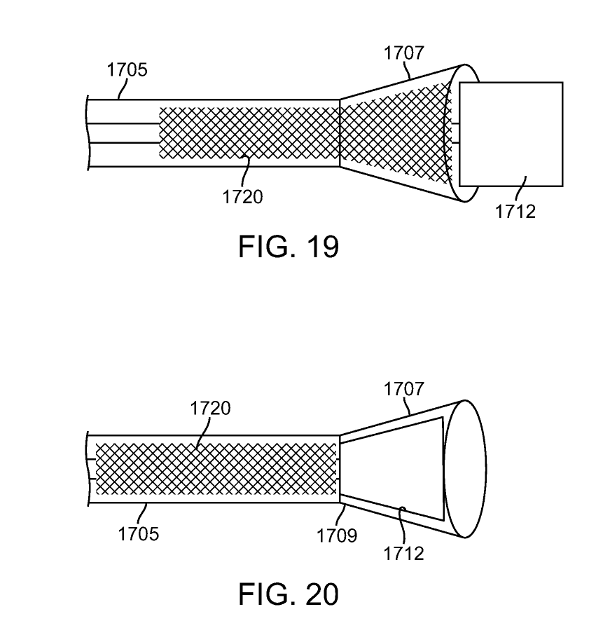

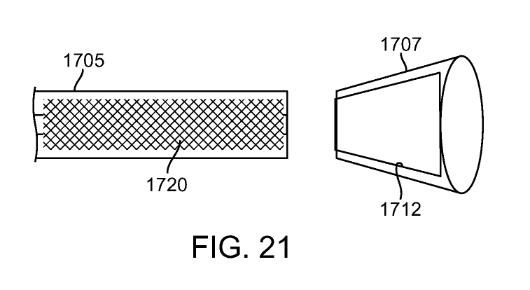

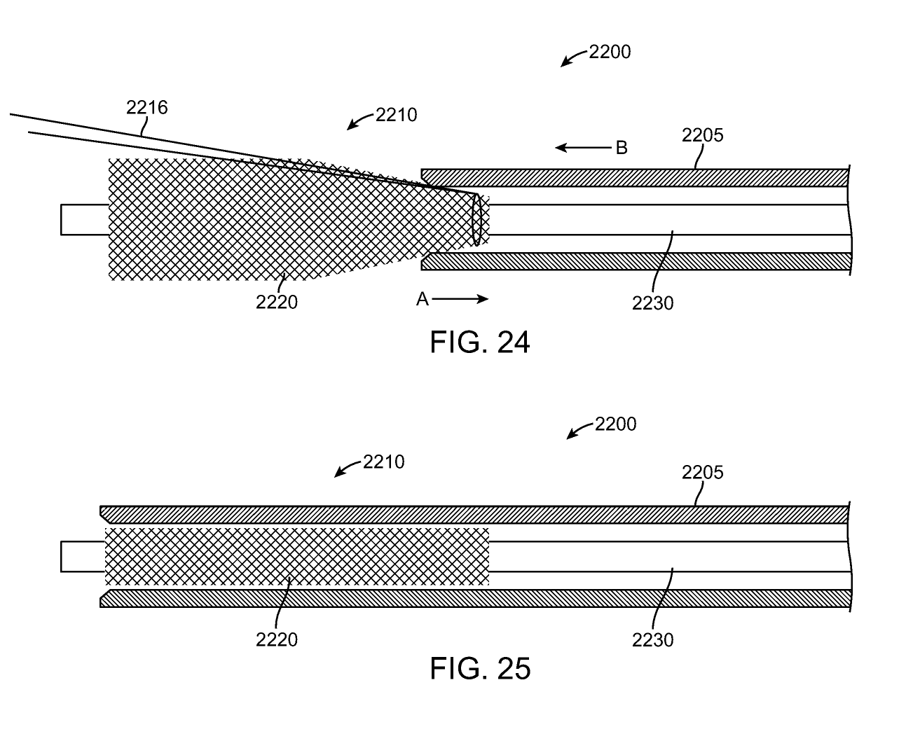

FIGS. 1 to 42 illustrate various embodiments of braided stent loading and delivery systems and methods of using the stent loading and delivery systems in accordance with the present invention. The various embodiments illustrated each show a braided stent delivery system having a stent loading assembly for loading a stent into a delivery catheter just prior to insertion into a patient's vascular system.

FIG. 1 is a side view of a stent loading and delivery system 100 made in accordance with the present invention. In this embodiment, the polymeric stent 120 is maintained outside of the delivery catheter until such time as it is loaded by a clinician just prior to use and insertion into a vessel. Stent delivery system 100 includes catheter 105, stent loading assembly 110 and stent 120. In this embodiment of a stent loading and delivery system, stent 120 is retained on stent loading assembly during storage and pre-delivery in an expanded non-compressed state. Stent loading assembly 110 is configured to move stent 120 into a compressed delivery configuration prior to insertion, as discussed below.

Catheter 105 comprises an elongated tubular member having a substantially circular cross-section and inside and outside walls that are substantially smooth. Catheter 105 is secured at its proximal end to a fitting and control mechanism 107 for controlling stent loading assembly 110. Catheter 105 may be manufactured from any suitable material such as, for example, a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® block copolymer, Vestamid® plastic resin, Tecoflex® thermoplastic polyurethanes, Halar® fluoropolymer, Hyflon® fluoropolymers, Pellathane® polyurethane, combinations thereof, and the like. Catheter 105 includes lumen 109 formed therethrough.

Stent 120 can be any variety of braided polymeric implantable prosthetic devices known in the art. In one embodiment, stent 120 is a self-expanding polymeric stent. Stent 120 includes a braided stent framework 122 having a first framework end 124 and a second framework end 126. The braided stent framework 122 is formed of a number of fibers 128 braided together to form a generally tubular body. Those skilled in the art will appreciate that the particular braid pattern can be selected as desired for a particular application. Those skilled in the art will also appreciate that the fibers 128 at the first framework end 124 and second framework end 126 can be free of each other or connected together as desired for a particular application.

The fibers 128 of the braided stent framework 122 can be made of a wide variety of medical implantable materials, such as nondegradable, bioabsorbable and biodegradable metals and polymers. In some embodiments, stent framework 122 may be a combination of nondegradable, biodegradable and bioabsorbable materials. The nondegradable polymer can be, for example, polyethylene naphthalate. The bioabsorbable polymer can be a homopolymer or copolymer of the monomers: glycolide, p-dioxanone, lactide, ε-caprolactone, or trimethylene carbonate (TMC), or any blend or ratio combination thereof. For example, poly(lactide-co-glycolide), poly(L-lactide), poly(L,DL, -lactide), poly(lactide-co-lactide-co-trimethylene carbonate), poly(lactide-co-caprolactone), poly(ε-caprolactone), or blends thereof. In one embodiment, the biodegradable metal is magnesium.

In one embodiment, the stent 120 can be capable of carrying a coating 125. In another embodiment, the stent 120 can include one or more therapeutic agents within the stent material. In one embodiment, coating 125 includes at least one therapeutic agent or drug. Throughout, the terms “therapeutic agent” and “drug” are used interchangeably and refer to any agent that is a “biologically or pharmacologically active substance” whether synthetic or natural that has a pharmacological, chemical, or biological effect on the body or a portion thereof. A therapeutic agent is capable of producing a beneficial effect against one or more conditions including coronary restenosis, cardiovascular restenosis, angiographic restenosis, arteriosclerosis, hyperplasia, and other diseases or conditions. The therapeutic agent may be, for example, anticoagulants, anti-inflammatories, fibrinolytics, antiproliferatives, antibiotics, therapeutic proteins, recombinant DNA products, bioactive agents, diagnostic agents, radioactive isotopes, and radiopaque substances.

Drug coating 125 containing at least one therapeutic agent may additionally contain excipients including solvents or other solubilizers, stabilizers, suspending agents, antioxidants, and preservatives, as needed to deliver an effective dose of the therapeutic agent to the treatment site. The therapeutic agent coating 125 may be applied by any means known in the art such as, for example, by spraying, dipping, brushing, and deposition. In one embodiment, the coating is applied as a liquid by brushing or spraying, and then dried to remove solvent using air, vacuum, or heat, and any other effective means of causing the formulation to adhere to the stent framework. If needed, drug coating 125 is cured by exposure to ultraviolet light, heat, gamma irradiation or any other appropriate means. In other embodiments, the therapeutic agent may be impregnated within the stent framework at varying depths such as, for example, full penetration or superficial penetration of the fibers composing the stent framework. In one embodiment, the therapeutic agent may be introduced during the extrusion process for forming fibers. In other embodiments the therapeutic agent is impregnated into the substrate forming the fibers after the fibers are formed or after the stent framework is formed.

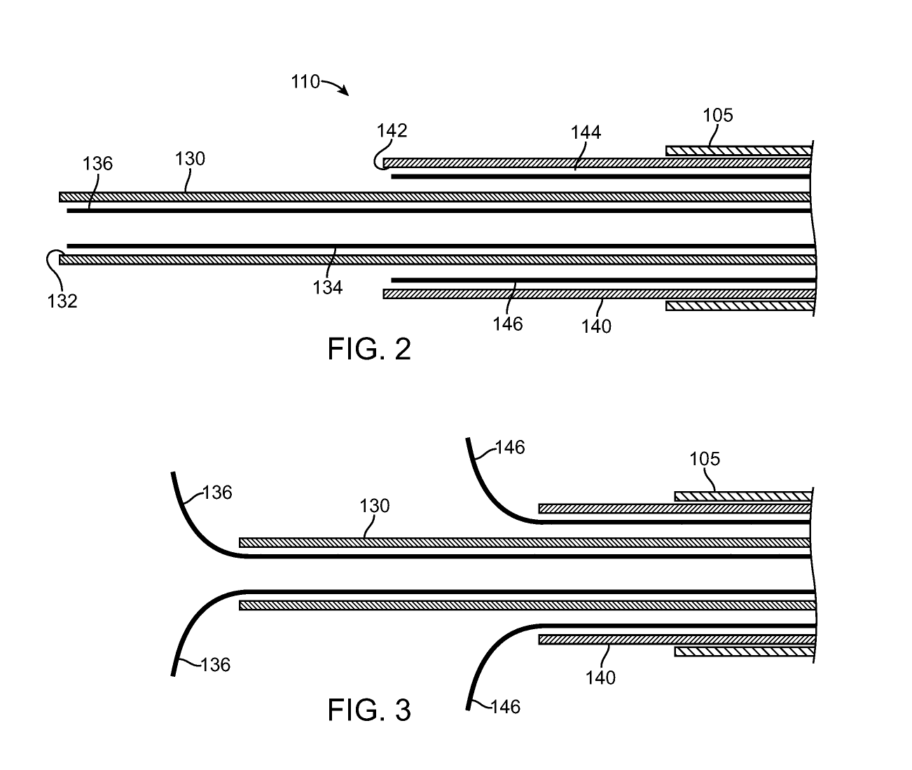

FIGS. 2 and 3 are detailed illustrations of stent loading assembly 110, located at the distal end of stent loading and delivery system 100, made in accordance with the present invention. Stent loading assembly 110 is disposed in catheter lumen 109. Stent loading assembly 110 includes inner sheath 130 having lumen 132 and outer sheath 140 having lumen 142. Stent loading assembly 110 further includes elongate distal hook members 134 disposed in lumen 132 and elongate proximal hook members 144 disposed in lumen 142. Inner sheath 130 and outer sheath 140 are concentrically arranged elongate tubular members that extend from control mechanism 107 to and beyond distal end 106 of catheter 105. The distance that each of inner sheath 130 and outer sheath 140 extends beyond the distal end of catheter 105 may be determined by the length of stent 120. As shown in FIG. 1, inner sheath 130 has a length to extend distal to a distal end of stent 120. Outer sheath 140 has a length to extend distal to a proximal end of stent 120.

Inner sheath 130 and outer sheath 140 may be composed of the same or similar materials as those listed above for catheter 105. In one embodiment, a distal portion of inner sheath 130 and outer sheath 140 is composed of a material having sufficient rigidity to maintain distal hooks 136 and proximal hooks 146 in a substantially straight delivery configuration as shown in FIG. 2.

Distal hook members 134 are elongated members having a proximal end that extends to control mechanism 107 and a distal end that terminates adjacent the distal end of stent 120. Proximal hook members 144 are elongated members having a proximal end that extends to control mechanism 107 and a distal end that terminates adjacent the proximal end of stent 120. Distal and proximal hook members 134, 144 can be made of a wide variety of metallic or polymeric materials and combinations thereof. The distal ends of each of distal and proximal hook members 134, 144 form distal hooks 136 and proximal hooks 146, respectively. Distal hooks 136 and proximal hooks 146 are composed of a shape memory material that when released from inner sheath 130 or outer sheath 140, respectively, forms a hook that releasably engages stent 120. FIG. 3 illustrates distal hooks 136 and proximal hooks 146 in a partially deployed position.

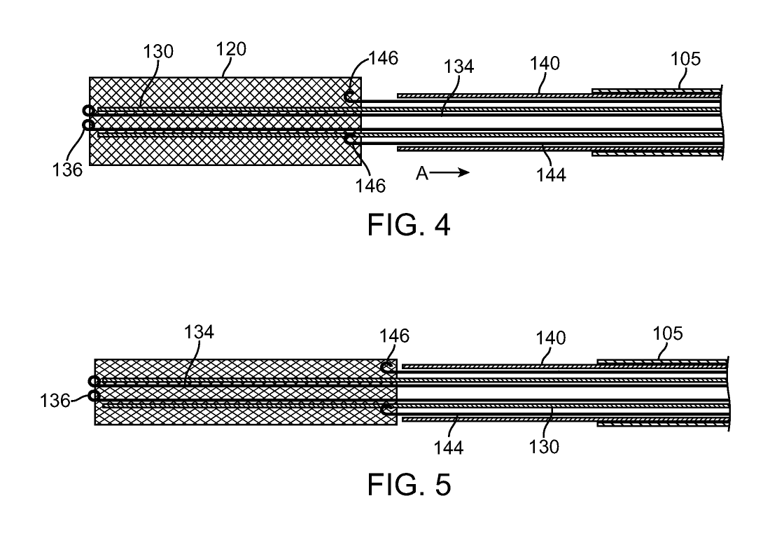

FIG. 4 is a detailed illustration of stent loading assembly 110 with stent 120 in a pre-compression loading configuration and FIG. 5 is a detailed illustration of stent loading assembly 110 with stent 120 in a compressed loaded delivery configuration. In use, a clinician begins to load stent 120 onto delivery catheter 105 by first retracting inner sheath 130 and outer sheath 140 in the direction of arrow A as seen in FIG. 4. Retraction of sheaths 130 and 140 releases the restrained distal and proximal hooks 136, 146 to allow hooks 136, 146 to move into the pre-set hook configuration. During the release of hooks 136, 146 the hooks curl around at least one fiber of stent 120. Sheaths 130, 140 may be withdrawn from hooks 136, 146 simultaneously or in series. Sheaths 130, 140 are withdrawn by the clinician by pulling on the proximal end of the respective sheaths operably attached to control mechanism 107. In another embodiment, the clinician releases the hooks 136, 146 by advancing the hooks in a distal direction while sheaths 130, 140 remain stationary.

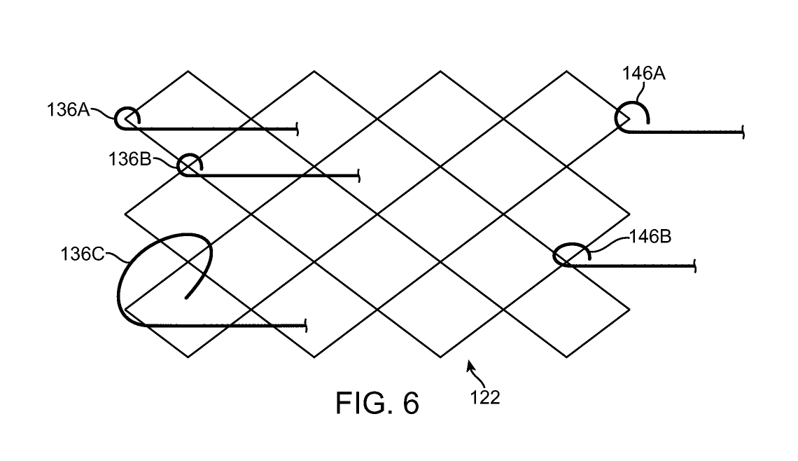

FIG. 6 illustrates exemplary placement of hooks 136 and 146 around stent fibers of stent framework 122. Hooks 136, 146 can be positioned to grasp any portion of the stent framework adjacent the respective hook. Hooks 136A and 146A curl around stent fibers at an end of stent framework 122. Hooks 136B and 146B curl around stent fibers at a junction of fibers of stent framework 122. Hook 136C curls around fibers at an end as well as fibers at a junction. Those with skill in the art will appreciate that the hooks can be positioned and released to curl around any portion and any number of fibers of stent framework 122. FIG. 6 illustrates the hooks having nearly circular tips once the hooks are deployed from their respective sheaths. In other embodiments, the radius of curvature of the hook is between 90 and 180 degrees. In other embodiments, the radius of curvature is between 180 and 360 degrees. Those with skill in the art will recognize that the curvature of the hooks may vary depending on such factors as, the stent material, the particular application and the presence or absence of a sheath such as sheath 805, discussed below.

Returning to FIG. 5, stent 120 is shown in a compressed delivery configuration. Stent 120 is moved into the compressed delivery configuration by moving at either the distal hook members, the proximal hook members or both in such a manner as to elongate and compress the stent into a delivery configuration. In one embodiment, stent 120 is moved into the delivery configuration by maintaining distal hook members 134 in a substantially static position while pulling on proximal hook members 144. In another embodiment, stent 120 is moved into the delivery configuration by maintaining proximal hook members 144 in a substantially static position while pushing on distal hook members 134. Those with skill in the art will appreciate that any combination of pushing and/or pulling of distal and proximal hook members 134, 144 may be used to place stent 120 into the desired delivery configuration. In one embodiment, once the desired compression is achieved, the inner and outer sheaths and/or the distal and proximal hook members may be locked into place at control handle 107.

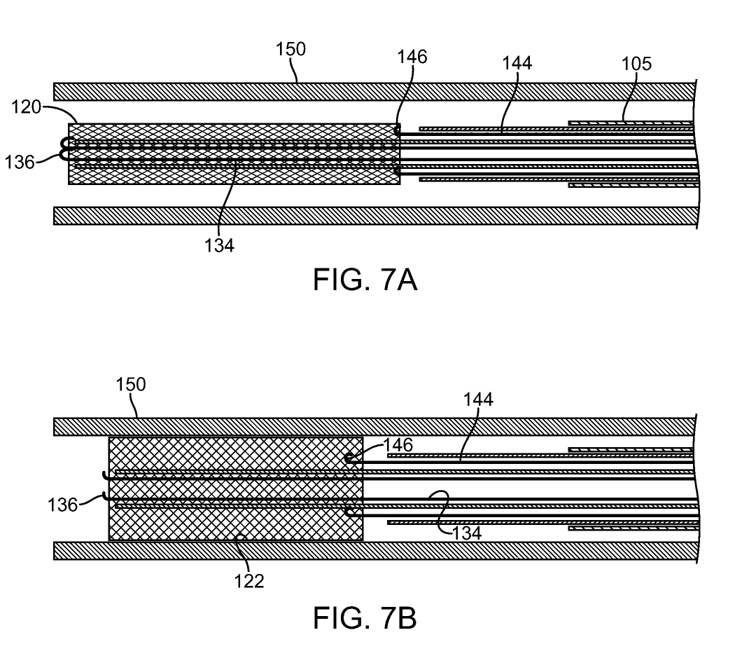

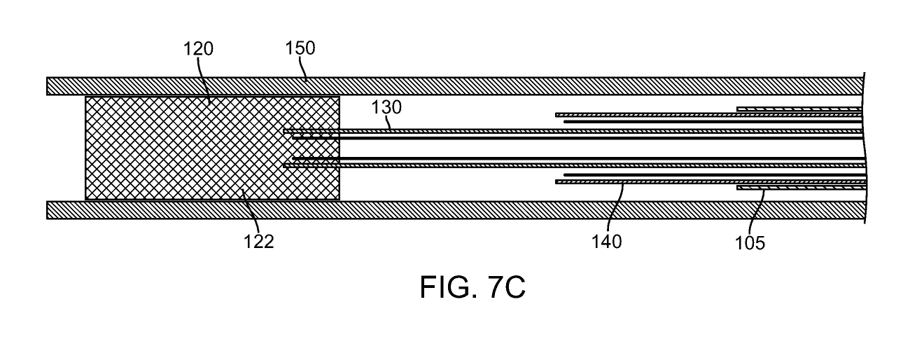

Referring to FIGS. 7A to 7C, FIG. 7A shows the compressed stent 120 advanced through the vessel 150 to the deployment site. Stent 120 is held in the compressed delivery configuration state by the spaced-apart distal and proximal hooks. Referring to FIG. 7B, the hooks are released from the stent framework to deploy stent 120 at the treatment site. In one embodiment, the hooks are released from the stent 120 by retracting the distal and proximal hook members 136, 146 into inner and outer sheaths 130, 140 respectively. Alternatively, inner and outer sheaths 130, 140 are advanced in a distal direction over the distal and proximal hook members to cover and restrain the distal and proximal hooks 136, 146. Once hooks 136 and 146 unfurl, the braided stent framework 122 expands toward the wall of vessel 150. The circumference of the braided stent framework 122 increases, firmly seating stent 120 in the vessel 150, as shown in FIG. 7C. Delivery catheter 105 may then be withdrawn.

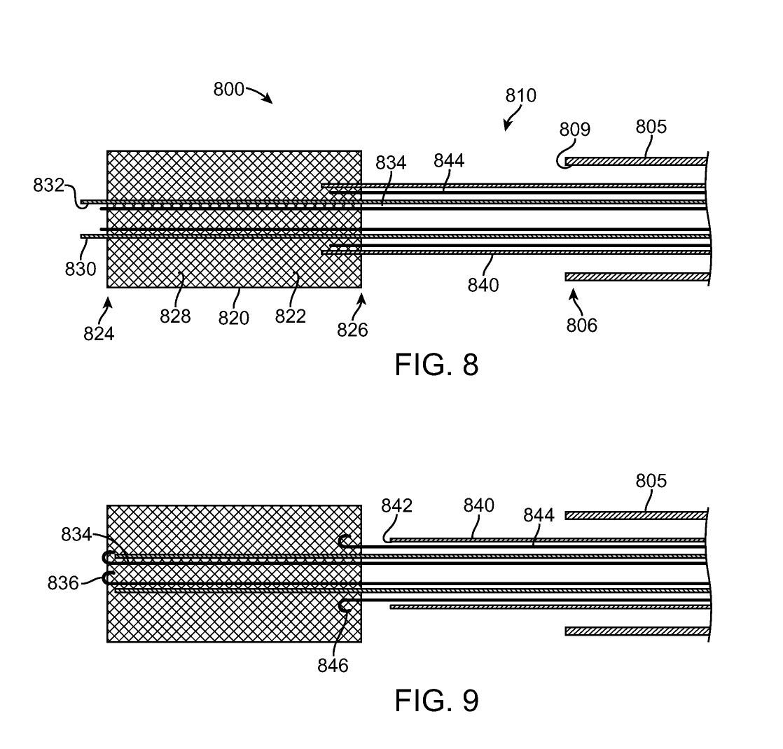

FIGS. 1 to 7C illustrate a stent loading and delivery system where the stent is delivered to the treatment site without being restrained within a sheath. FIGS. 8 to 11 illustrate one embodiment of a stent loading and delivery system 800 that utilizes a delivery sheath to encapsulate the stent for delivery. As with system 100, a stent delivered with system 800 is loaded just prior to insertion into the patient.

Stent loading and delivery system 800 is similar in many respects to system 100. Those aspects which are the same will not be discussed in detail. System 800 includes catheter 805, stent loading assembly 810 and stent 820. In this embodiment of a stent loading and delivery system, stent 820 is retained on stent loading assembly during storage and pre-delivery in an expanded non-compressed state. Stent loading assembly 810 is configured to move stent 820 into a compressed delivery configuration prior to insertion, as discussed below.

Catheter 805 comprises an elongated tubular member having a substantially circular cross-section and inside and outside walls that are substantially smooth. Catheter 805 is composed of materials that are the same as or similar to those discussed above for catheter 105.

Stent 820 can be any variety of braided polymeric implantable prosthetic devices known in the art. In one embodiment, stent 820 is a self-expanding polymeric stent. Stent 820 includes a braided stent framework 822 having a first framework end 824 and a second framework end 826. The braided stent framework 822 is formed of a number of fibers 828 braided together to form a generally tubular body. Stent 820 is the same as or similar to stent 120 discussed above. Stent 820 is composed of material that is the same as or similar to stent 120 discussed above. In one embodiment, stent 820 includes a coating the same as or similar to coating 125, as described above. In another embodiment, stent 820 includes one or more therapeutic agents within the stent material, as described above.

Stent loading assembly 810, located at the distal end of stent loading and delivery system 800, is similar to stent loading assembly 110. Stent loading assembly 810 is disposed in catheter lumen 809. Stent loading assembly 810 includes inner sheath 830 having lumen 832 and outer sheath 840 having lumen 842. Stent loading assembly 810 further includes elongate distal hook members 834 disposed in lumen 832 and elongate proximal hook members 844 disposed in lumen 842. Inner sheath 830 and outer sheath 840 are concentrically arranged elongate tubular members that extend from control mechanism 107 to and beyond distal end 806 of catheter 805. The distance that each of inner sheath 830 and outer sheath 840 extends beyond the distal end of catheter 805 may be determined by the length of stent 820. As shown in FIG. 8, inner sheath 830 has a length to extend distal to a distal end of stent 820. Outer sheath 840 has a length to extend distal to a proximal end of stent 820.

Inner sheath 830 and outer sheath 840 may be composed of the same or similar materials as those listed above for catheter 805. In one embodiment, a distal portion of inner sheath 830 and outer sheath 840 is composed of a material having sufficient rigidity to maintain distal hooks 836 and proximal hooks 846 in a substantially straight delivery configuration as shown in FIG. 8. Distal hook members 834 and proximal hook members 844 are composed of the same or similar materials as described above for hooks members 134 and 144. Distal hooks 836 and proximal hooks 846 are composed of shape memory material that assumes a preset shape upon release from sheaths 830, 840, respectively. The arrangement of sheaths, 830, 840 and hook members 834, 844 is the same as that described above for system 100.

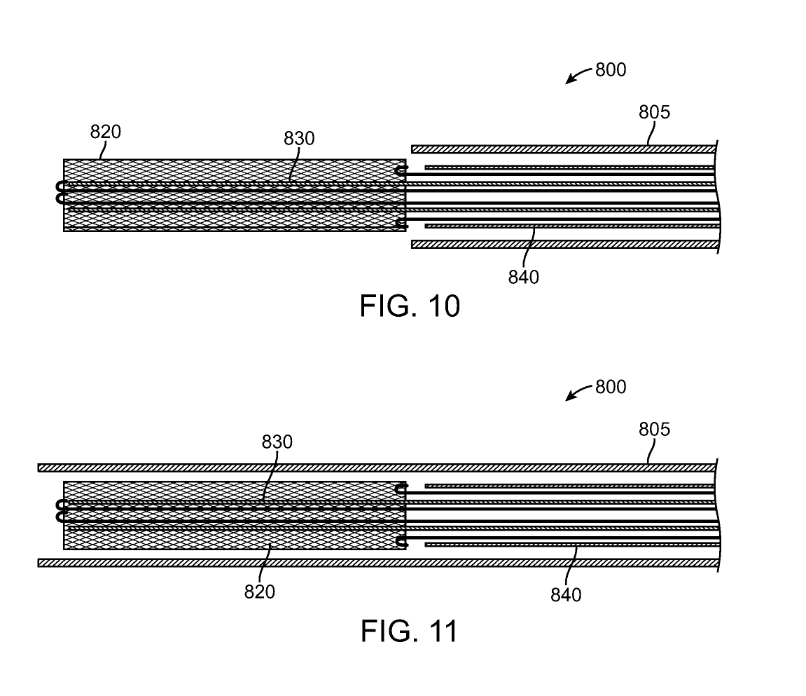

FIGS. 9 to 11 illustrate the loading of stent 820 into delivery catheter 805. Stent 820 is grasped and held by distal hooks 836 and proximal hooks 846 in a manner the same as that described above for system 100. Retraction of inner sheath 830 releases distal hooks 836 allowing the hooks to curl around and grasp at least one fiber of stent framework 822 at a distal end of stent 820. Similarly, retraction of outer sheath 840 releases proximal hooks 846 allowing the hooks to curl around and grasp at least one fiber of stent framework 822 at a proximal end of stent 820, as shown in FIG. 9. FIG. 10 illustrates the compression of stent 820 into a delivery configuration. In this embodiment, stent 820 is compressed into the delivery configuration in a manner similar to that described above for system 100. Stent 820 is moved into the compressed delivery configuration by moving at either the distal hook members, the proximal hook members or both in such a manner as to elongate and compress the stent into a delivery configuration. In one embodiment, stent 820 is moved into the delivery configuration by maintaining distal hook members 834 in a substantially static position while pulling on proximal hook members 844. In another embodiment, stent 820 is moved into the delivery configuration by maintaining proximal hook members 844 in a substantially static position while pushing on distal hook members 834. Those with skill in the art will appreciate that any combination of pushing and/or pulling of distal and proximal hook members 834, 844 may be used to place stent 820 into the desired delivery configuration. Once stent 820 is moved to the elongated and compresses delivery configuration, loading assembly 810 and stent 820 is drawn into delivery catheter 805, as shown in FIG. 11, for insertion into a vascular system. Once stent 820 is delivered to the treatment site, the loaded stent is positioned within the lesion. Delivery catheter 805 is then retracted to expose the stent for release from the loading assembly 810 of delivery catheter 805. In one embodiment, stent 820 is released from loading assembly 810 in a manner the same as, or similar to, the method described above for stent 120. Once stent 820 is deployed, catheter 805 is withdrawn from the patient.

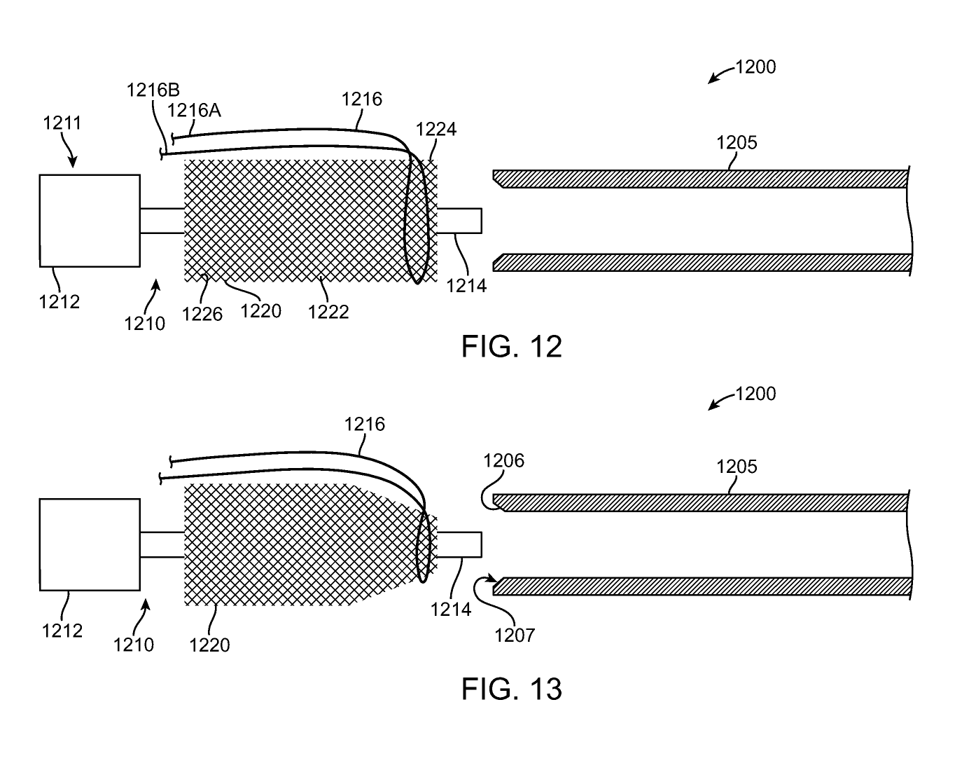

FIGS. 12 to 16 illustrate another embodiment of a stent loading and delivery system 1200 that utilizes a delivery sheath to encapsulate the stent for delivery. As with systems 100 and 800, a stent delivered with system 1200 is loaded just prior to insertion into the patient.

System 1200 includes delivery catheter 1205, stent loading assembly 1210 and stent 1220. Catheter 1205 comprises an elongated tubular member having a substantially circular cross-section and inside and outside walls that are substantially smooth. Catheter 1205 is composed of materials that are the same as or similar to those discussed above for catheter 105.

Stent 1220 can be any variety of braided metallic or polymeric implantable prosthetic devices known in the art. In one embodiment, stent 1220 is a self-expanding polymeric stent. Stent 1220 includes a braided stent framework 1222 having a first framework end 1224 and a second framework end 1226. Stent 1220 is the same as or similar to stent 120 discussed above. Stent 1220 is composed of material that is the same as or similar to stent 120 discussed above. In one embodiment, stent 1220 includes a coating the same as or similar to coating 125, as described above. In another embodiment, stent 1220 includes one or more therapeutic agents within the stent material, as described above.

Stent loading assembly 1210 includes a pushrod 1211 having an elongated rod portion 1214 extending from a handle 1212. Stent loading assembly 1210 also includes a cinch 1216. Cinch 1216 is an elongated filament that may be composed of a metallic or polymer material. Cinch 1216 is a drawstring having a first end 1216A and a second end 1216B. Cinch 1216 is looped around the first end 1224 of stent 1220, as shown in FIG. 12.

Stent 1220 is loaded into lumen 1206 of delivery catheter 1205 by compressing first stent end 1224 into contact with rod 1214. First stent end 1224 is crimped onto rod 1214 by pulling on cinch ends 1216A and 1216B thereby reducing the diameter of stent end 1224. Rod 1214 with reduced diameter stent end 1224 is then inserted into the open end 1207 of catheter 1205, as shown in FIG. 14. In one embodiment, the receiving end of delivery catheter 1205 is chamfered on the inner diameter of opening 1207 to facilitate loading of stent 1220.

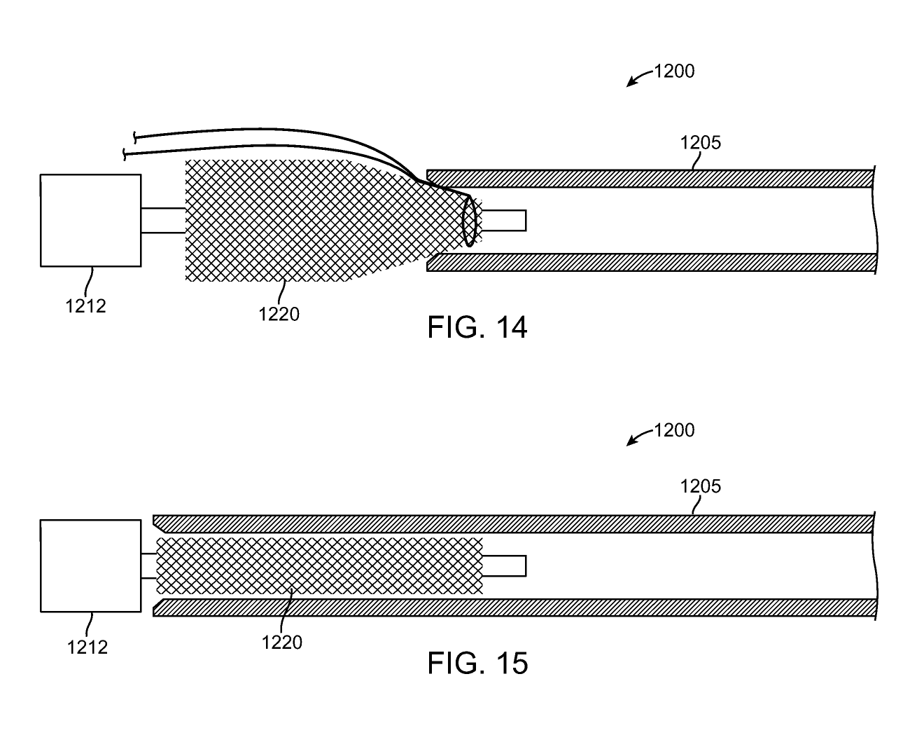

Pushrod 1211 is pushed toward catheter end 1207 until stent 1220 is fully inserted. Continued insertion of pushrod 1211 reduces the diameter of stent 1220 along its entire length as is shown in FIG. 15. Once stent 1220 is fully inserted, the clinician pulls on one of cinch end 1216A or 1216B to withdraw cinch 1216 from around stent end 1224. Upon the complete withdrawal of cinch 1216, rod 1214 can be withdrawn by pulling on handle 1212 leaving stent 1220 within delivery catheter 1205 as shown in FIG. 16. Delivery catheter 1205 with loaded stent 1220 may then be inserted into the patient and advanced to the treatment site as described above. Stent 1220 is deployed at the treatment site in any manner as is known in the art. Once stent 1220 is deployed, catheter 1205 is withdrawn from the patient.

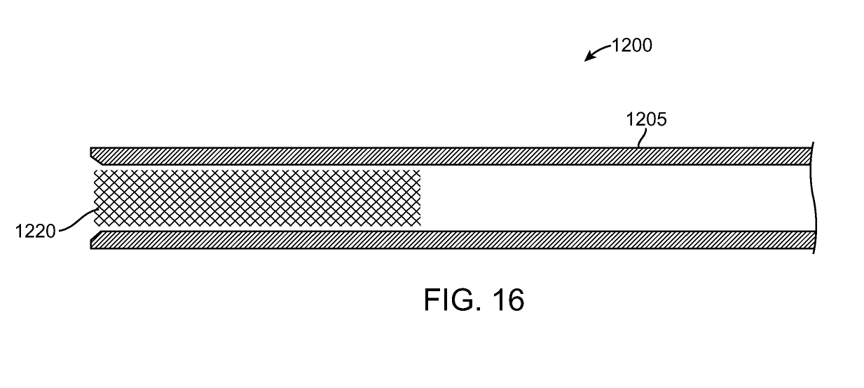

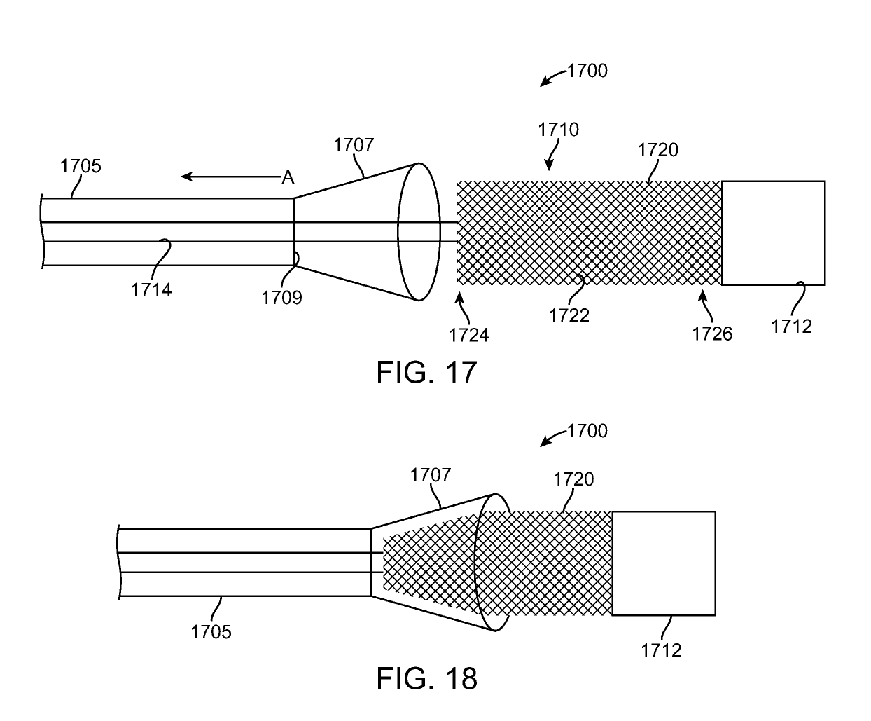

FIGS. 17-21 illustrate another embodiment of a stent loading and delivery system 1700 that utilizes a delivery sheath to encapsulate the stent for delivery. As with systems 100, 800 and 1200, a stent delivered with system 1700 is loaded just prior to insertion into the patient.

System 1700 includes delivery catheter 1705, stent loading assembly 1710 and stent 1720. Catheter 1705 comprises an elongated tubular member having a substantially circular cross-section and inside and outside walls that are substantially smooth. Catheter 1705 is composed of materials that are the same as or similar to those discussed above for catheter 105. Catheter 1705 includes a detachable funnel 1707 for facilitating insertion of stent 1720 into delivery catheter 1705. Funnel 1707 may be removably attached to catheter 1705 in any suitable manner. In one embodiment, the junction 1709 between funnel 1707 and the distal end of catheter 1705 is perforated. In another embodiment, funnel 1707 may be attached to catheter 1705 by an adhesive.

Stent 1720 can be any variety of braided metallic or polymeric implantable prosthetic device known in the art. In one embodiment, stent 1720 is a self-expanding polymeric stent. Stent 1720 includes a braided stent framework 1722 having a first framework end 1724 and a second framework end 1726. Stent 1720 is the same as or similar to stent 120 discussed above. Stent 1720 is composed of material that is the same as or similar to stent 120 discussed above. In one embodiment, stent 1720 includes a coating the same as or similar to coating 125, as described above. In another embodiment, stent 1720 includes one or more therapeutic agents within the stent material, as described above.

Stent loading assembly 1710 includes inner member 1714 and collapsible stopper 1712 releasably attached to a distal end of inner member 1714 and abutting stent end 1726 of stent 1720. Collapsible stopper 1712 is composed of any suitable material such as, but not limited to, a compressible rubber, a thermoplastic elastomeric rubber, compressible plastic of a suitable durometer. In other embodiments, collapsible stopper 1712 is composed of Pebax® Plastic, Kraton® thermoplastic rubber and Hytrel® polyester elastomer. In this embodiment, stent 1720 is loaded into catheter 1705 by moving inner member 1714 in the direction of arrow A. As inner member 1714 is translated, stent 1720 is drawn into funnel 1707. The shape of funnel 1707 reduces the diameter of stent 1720 and compresses stent 1720 about inner member 1714. Inner member 1714 is drawn into catheter 1705 until the entire length of stent 1720 is within catheter 1705 and collapsible stopper 1712 is within funnel 1707, as shown in FIG. 20. Once the stent is loaded, funnel 1707 with stopper 1712 is removed from catheter 1705 by breaking the perforation bond or adhesive bond by which funnel 1707 was attached to catheter 1705. Delivery catheter 1705 with loaded stent 1720 may then be inserted into the patient and advanced to the treatment site as described above. Stent 1720 is released at the treatment site in any manner known in the art. Once stent 1720 is deployed, catheter 1705 is withdrawn from the patient.

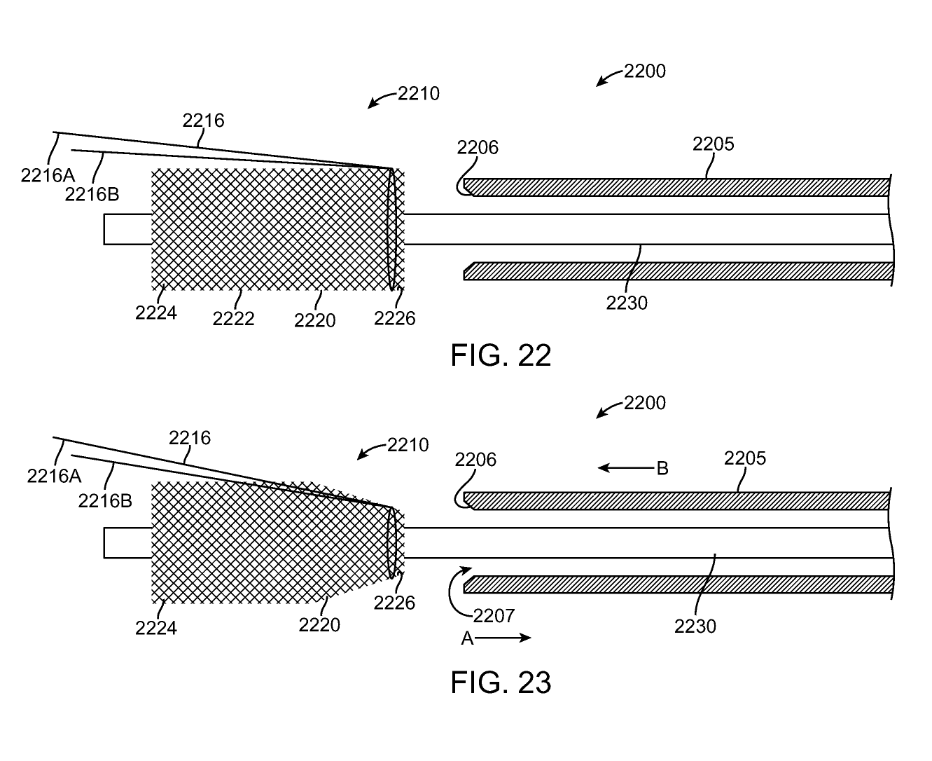

FIGS. 22 to 25 illustrate a series of side views for another embodiment of a stent loading and delivery system 2200 that utilizes a delivery sheath to encapsulate the stent for delivery. System 2200 includes delivery catheter 2205, stent loading assembly 2210 and stent 2220. As with the systems described above, a stent delivered with system 2200 is loaded just prior to insertion into the patient. Catheter 2205 comprises an elongated tubular member having a substantially circular cross-section and inside and outside walls that are substantially smooth. Catheter 2205 is composed of materials that are the same as or similar to those discussed above for catheter 105.

Stent 2220 can be any variety of braided metallic or polymeric implantable prosthetic device known in the art. In one embodiment, stent 2220 is a self-expanding polymeric stent. Stent 2220 includes a braided stent framework 2222 having a first framework end 2224 and a second framework end 2226. Stent 2220 is the same as or similar to stent 120 discussed above. Stent 2220 is composed of material that is the same as or similar to stent 120 discussed above. In one embodiment, stent 2220 includes a coating the same as or similar to coating 125, as described above. In another embodiment, stent 2220 includes one or more therapeutic agents within the stent material, as described above.

Stent loading assembly 2210 includes inner member 2230 and cinch 2216. Cinch 2216 is an elongated filament that may be composed of a metallic or polymer material. Cinch 2216 is a drawstring having a first stent end 2216A and a second stent end 2216B. In this embodiment, cinch 2216 is looped around the second end 2226 of stent 2220, as shown in FIG. 22.

Stent 2220 is loaded into lumen 2206 of delivery catheter 2205 by compressing second stent end 2226 into contact with inner member 2230. Second stent end 2226 is crimped onto inner member 2230 by pulling on cinch ends 2216A and 2216B thereby reducing the diameter of second stent end 2226. Inner member 2230 with reduced diameter second stent end 2226 is then inserted into the open end 2207 of catheter 2205, as shown in FIG. 23, by pulling inner member 2230 in the direction of arrow A. In another example, the stent can be inserted into the open end 2207 of catheter 2205 by moving catheter shaft 2205 in a direction of arrow B while maintaining inner member in a substantially stationary position. In another embodiment, a combination of movements of the inner member and the outer catheter may be employed to insert the stent. In one embodiment, the receiving end of delivery catheter 2205 is chamfered on the inner diameter of opening 2207 to facilitate loading of stent 2220.

Inner member 2230 is pulled in the direction of arrow A until stent 2220 is fully inserted within the distal end of delivery catheter 2205. Continued translation of inner member 2230 reduces the diameter of stent 2220 along its entire length as is shown in FIG. 25. Once stent 2220 is fully inserted, the clinician pulls on one of cinch end 2216A or 2216B to withdraw cinch 2216 from around second stent end 2226. Delivery catheter 2205 with loaded stent 2220 may then be inserted into the patient, advanced to the treatment site and released as is known in the art.

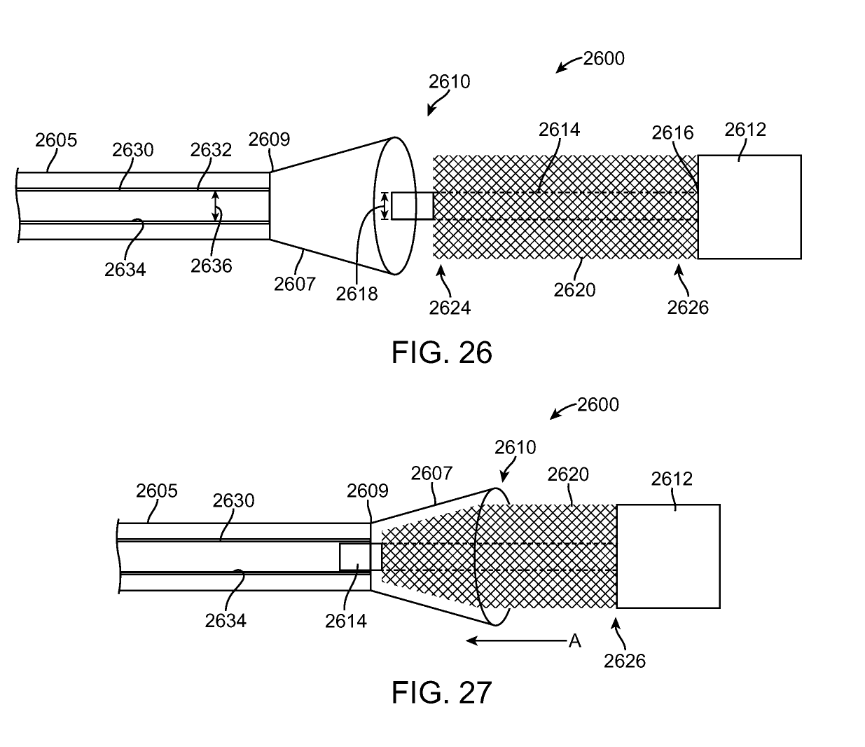

FIGS. 26-29 illustrate another embodiment of a stent loading and delivery system 2600 that utilizes a delivery sheath to encapsulate the stent for delivery. As with the systems described above, a stent delivered with system 2600 is loaded into the delivery catheter just prior to insertion into the patient. The stent loading assembly of system 2600 is similar to stent loading assembly 1710, described above. However, in this embodiment, an elongate member is sized to fit within a lumen of an inner catheter, described in more detail below.

System 2600 includes delivery catheter 2605, stent loading assembly 2610 and stent 2620. Catheter 2605 comprises an elongated tubular member having a substantially circular cross-section and inside and outside walls that are substantially smooth. Catheter 2605 is composed of materials that are the same as or similar to those discussed above for catheter 105. System 2600 also includes an inner catheter 2630. Inner catheter 2630 and catheter 2605 are concentrically arranged about a common axis. Inner catheter 2630 is composed of the same or similar materials as catheter 2605. Inner catheter 2630 has a wall 2632 defining a lumen 2634, the lumen having a diameter 2636. Lumen 2634 is sized to receive an inner member 2614 of stent loading assembly 2610.

Catheter 2605 includes a detachable funnel 2607 for facilitating insertion of stent 2620 into delivery catheter 2605. Funnel 2607 is removably attached to a distal end of catheter 2605 in any suitable manner. In one embodiment, the junction 2609 between funnel 2607 and the distal end of catheter 2605 is perforated. In another embodiment, funnel 2607 may be attached to catheter 2605 by an adhesive.

Stent 2620 can be any variety of braided metallic or polymeric implantable prosthetic device known in the art. In one embodiment, stent 2620 is a self-expanding polymeric stent. Stent 2620 includes a braided stent framework 2622 having a first framework end 2624 and a second framework end 2626. Stent 2620 is the same as or similar to stent 120 discussed above. Stent 2620 is composed of material that is the same as or similar to stent 120 discussed above. In one embodiment, stent 2620 includes a coating the same as or similar to coating 125, as described above. In another embodiment, stent 2620 includes one or more therapeutic agents within the stent material, as described above.

Stent loading assembly 2610 includes inner member 2614 and collapsible stopper 2612. Stopper 2612 is attached to an end 2616 of inner member 2614 and abuts stent end 2626 of stent 2620. Inner member 2614 has a complementary shape to lumen 2634 and has an outer diameter 2618 that is less than inner diameter 2636 of lumen 2634 such that inner member 2614 fits within lumen 2634. Stopper 2612 is similar to or the same as collapsible stopper 1712, described above.

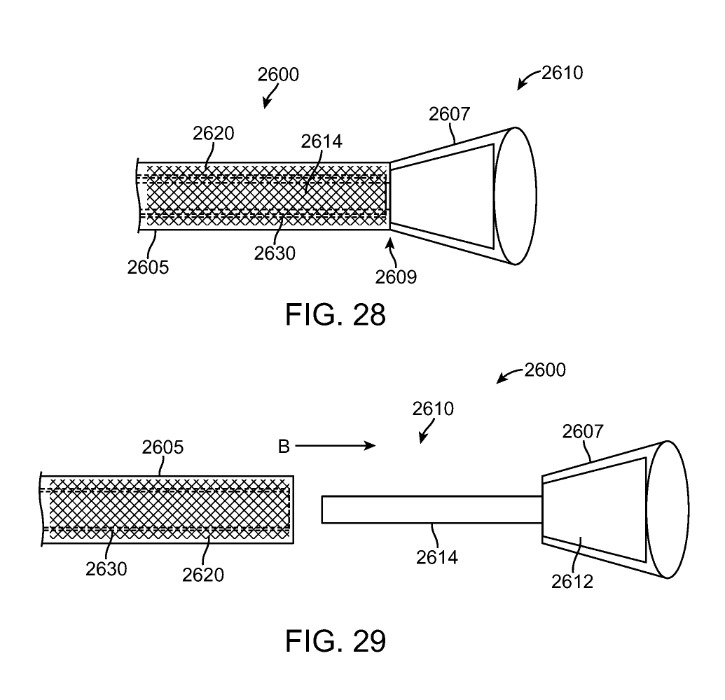

In this embodiment, stent 2620 is loaded into catheter 2605 by moving stent loading assembly 2610 in the direction of arrow A to insert inner member 2614 into lumen 2634. As stent loading assembly 2610 is translated in the direction of arrow A, stent 2620 is moved into contact with the inner surface of funnel 2607. With continued translation, the diameter of stent 2620 is reduced as the inner diameter of the funnel 2607 decreases. The diameter of stent 2620 decreases and compresses stent 2620 about inner catheter 2630. Inner member 2614 is moved into inner catheter 2630 until the entire length of stent 2620 is within catheter 2605 and collapsible stopper 2612 is within funnel 2607, as shown in FIG. 28. Once the stent is loaded, funnel 2607 with stopper 2612 and inner member 2614 is removed from catheter 2605 by breaking the perforation bond or adhesive bond by which funnel 2607 was attached to catheter 2605 and moving the assembly in the direction of arrow B, as shown in FIG. 29. Delivery catheter 2605 with loaded stent 2620 may then be inserted into the patient and advanced to the treatment site as described above. Stent 2620 is released at the treatment site in any manner known in the art. Once stent 2620 is deployed, catheter 2605 is withdrawn from the patient.

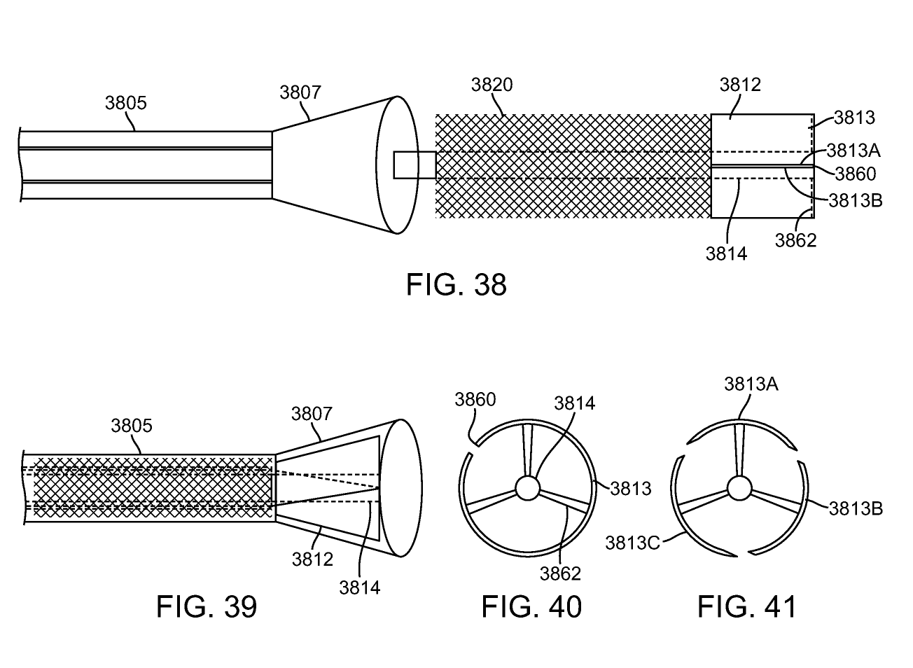

FIGS. 38 and 39 illustrate another embodiment of a collapsible stopper 3812 that may be used with system 2600. In this embodiment, the collapsible stopper 3812 comprises a sheet 3813 of thin walled elastomeric material disposed around inner member 3814. As shown in FIG. 38 and FIG. 40, the sheet 3813 of elastomeric material is not completely wrapped around inner member 3814 leaving a gap or slit 3860 between sheet edges 3813A and 3813B. This separation facilitates the collapse of stopper 3812 when the stent 3820 is inserted into catheter 3805 through funnel 3807. FIG. 40 shows that the sheet of material 3813 is attached to inner member 3814 by at least one, in this case three, radial connectors 3862. Radial connectors 3862 may be the same or similar material as sheet 3813. Radial connectors may be composed of a collapsible material. FIG. 41 illustrates another embodiment of a collapsible stopper. FIG. 41 also shows that in one embodiment, stopper 3812 is composed of three sheets of material sheet 3813A, 3813B, and 3813C. In this or other embodiments, the edges of sheet(s) 3813 may be tapered to further facilitate the collapse of stopper 3812 as the stopper enters funnel 3807.

In another embodiment, system 2600 does not include a funnel at the distal end of the delivery catheter 2605. In this embodiment, the distal end of the delivery catheter is comprises a flexible tip. This flexible tip is composed of a material that expands to receive and accommodate stent 2620 as it is inserted into the distal end of the delivery catheter.

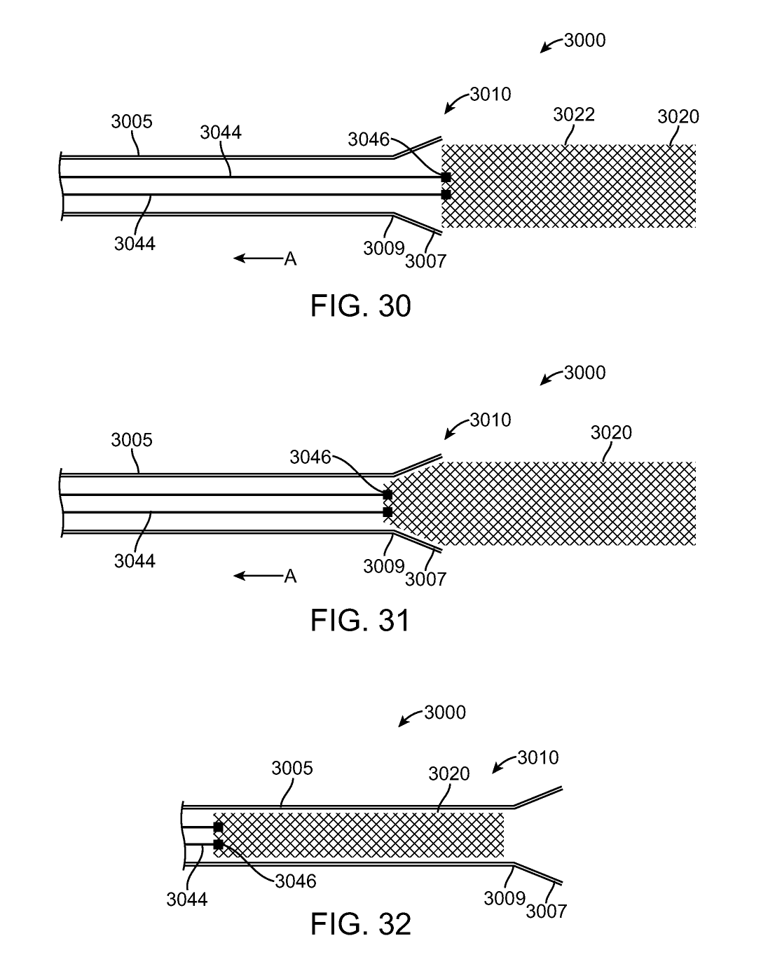

FIGS. 30-32 illustrate another embodiment of a stent loading and delivery system 3000 that utilizes a delivery sheath to encapsulate the stent for delivery. As with the systems described above, a stent delivered with system 3000 is loaded into the delivery catheter just prior to insertion into the patient.

System 3000 includes delivery catheter 3005, stent loading assembly 3010 and stent 3020. Catheter 3005 comprises an elongated tubular member having a substantially circular cross-section and inside and outside walls that are substantially smooth. Catheter 3005 is composed of materials that are the same as or similar to those discussed above for catheter 105.

Catheter 3005 includes a detachable funnel 3007 for facilitating insertion of stent 3020 into delivery catheter 3005. Funnel 3007 is removably attached to a distal end of catheter 3005 in any suitable manner. In one embodiment, the junction 3009 between funnel 3007 and the distal end of catheter 3005 is perforated. In another embodiment, funnel 3007 may be attached to catheter 3005 by an adhesive.

Stent 3020 can be any variety of braided metallic or polymeric implantable prosthetic device known in the art. In one embodiment, stent 3020 is a self-expanding polymeric stent. Stent 3020 includes a braided stent framework 3022 having a first framework end 3024 and a second framework end 3026. Stent 3020 is the same as or similar to stent 120 discussed above. Stent 3020 is composed of material that is the same as or similar to stent 120 discussed above. In one embodiment, stent 3020 includes a coating the same as or similar to coating 125, as described above. In another embodiment, stent 3020 includes one or more therapeutic agents within the stent material, as described above.

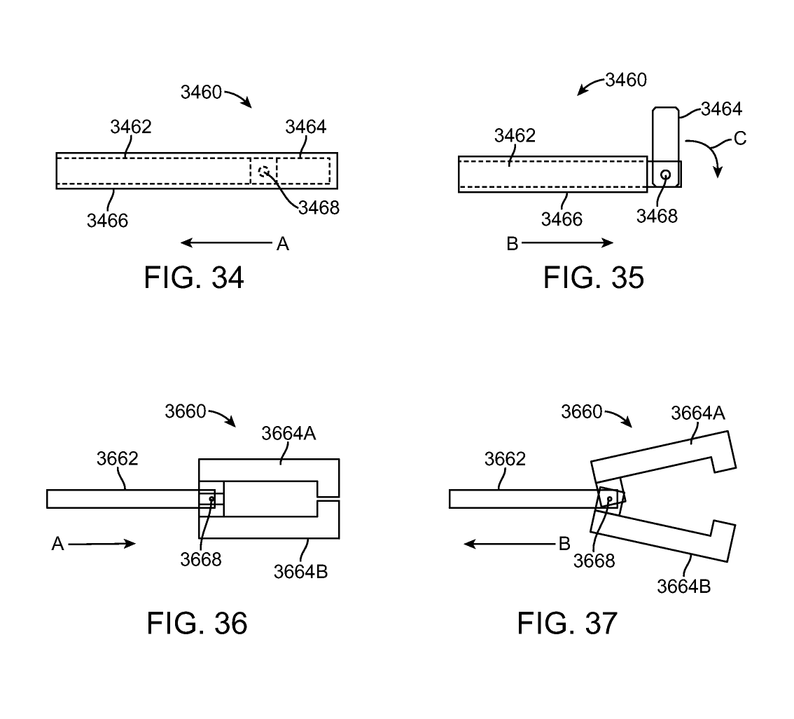

Stent loading assembly 3010 includes at least one elongate hook member 3044. Each elongate hook member 3044 includes a hook 3046 for grasping the stent framework 3022 of stent 3020. In this embodiment, system 3000 includes two elongate hook members 3044 for grasping the stent framework 3022. In one embodiment, hook members 3044 may be a hook member having a distal shape memory hook configured as described above. In another embodiment, hook members 3044 include a mechanical hook or grasping portion, as described in more detail below and shown in FIGS. 34 to 37.

In this embodiment, hooks 3046 are engaged with stent framework 3220 and stent 3020 is loaded into catheter 3005 by moving hook members 3044 of stent loading assembly 3010 in the direction of arrow A. As hook members 3044 are translated in the direction of arrow A, stent 3020 is moved into contact with the inner surface of funnel 3007. With continued translation, the diameter of stent 3020 is reduced as the inner diameter of the funnel 3007 decreases. The diameter of stent 3020 decreases and compresses stent 3020 to fit within the lumen of catheter 3005. Hook members 3044 are translated until the entire length of stent 3020 is within catheter 3005, as shown in FIG. 32. Once the stent is loaded, funnel 3007 is removed from catheter 3005 by breaking the perforation bond or adhesive bond by which funnel 3007 was attached to catheter 3005. Delivery catheter 3005 with loaded stent 3020 may then be inserted into the patient and advanced to the treatment site as described above. Stent 3020 is released at the treatment site in any manner known in the art. Once stent 3020 is deployed, catheter 3005 is withdrawn from the patient.

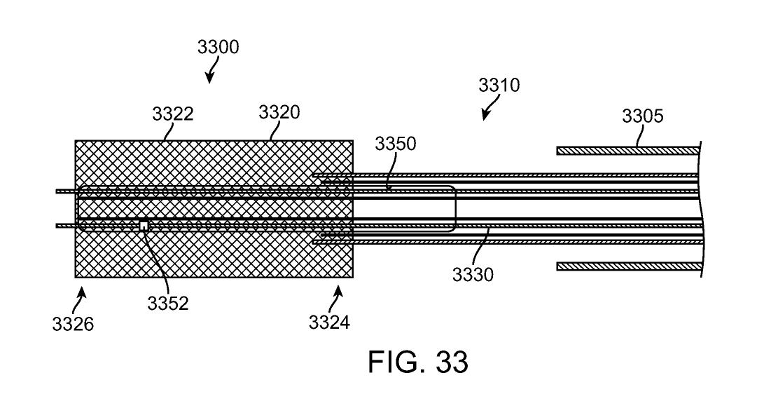

FIG. 33 illustrates another embodiment of a stent loading and delivery system 3300. As with the systems described above, a stent delivered with system 3300 is loaded into or onto the delivery catheter just prior to insertion into the patient. System 3300 is the same as or similar to many aspects of systems 100 and 800 described above. In this embodiment, system 3300 includes a delivery balloon 3350 to aid in the delivery of the stent 3320 at the treatment site. Both system 100 and 800 may be modified to include a delivery balloon disposed on the inner catheter 3330 that is inflated through inflation port 3352.

System 3300 includes delivery catheter 3305, stent loading assembly 3310 and stent 3320. Catheter 3305 comprises an elongated tubular member having a substantially circular cross-section and inside and outside walls that are substantially smooth. Catheter 3305 is composed of materials that are the same as or similar to those discussed above for catheter 105.

Stent 3320 can be any variety of braided metallic or polymeric implantable prosthetic device known in the art. In one embodiment, stent 3320 is a self-expanding polymeric stent. Stent 3320 includes a braided stent framework 3322 having a first framework end 3324 and a second framework end 3326. Stent 3320 is the same as or similar to stent 120 discussed above. Stent 3320 is composed of material that is the same as or similar to stent 120 discussed above. In one embodiment, stent 3320 includes a coating the same as or similar to coating 125, as described above. In another embodiment, stent 3320 includes one or more therapeutic agents within the stent material, as described above.

Stent loading assembly 3010 includes the same, or similar, structures and elements described above for systems 100 and 800 and will not be described further. As discussed above, stent loading assembly includes a balloon 3350 for aiding in the delivery of the stent 3320 at the treatment site. In use, the stent is loaded as described above for system 100 or system 800 and is delivered to the treatment site as described above. In addition, balloon 3350 may be inflated after the hooks have been released from the stent framework 3322 to expand stent 3320 into contact with the lesion and/or vessel wall. Once stent 3320 is deployed, balloon 3350 is deflated and catheter 3305 is withdrawn from the patient.

FIGS. 34 to 37 illustrate mechanical hook assemblies 3460, 3660 made in accordance with the present invention. Each of the systems described above that include the use of hooks to grasp the stent framework may be modified to comprise a mechanical hook in addition to or in replacement of the shape memory hooks illustrated. Hook assemblies 3460 (FIGS. 34 and 35) and 3660 (FIGS. 36 and 37) use mechanical means to operate. Referring to FIGS. 34 and 35, hook assembly 3460 comprises an elongate hook member 3462, a spring loaded hook 3464 and restraining sheath 3466. Hook 3464 is operably connected to hook member 3462 by spring 3468. In use, a distal end of hook assembly 3460 is positioned adjacent the stent framework and restraining sheath 3466 is withdrawn in the direction of arrow A to release hook 3464 and allow hook 3464 to move into the position shown in FIG. 35. Movement of sheath 3466 in the direction of arrow B moves hook 3464 in the direction of arrow C and into an essentially straight configuration that can be retrained by covering with sheath 3466.

Referring to FIGS. 36 and 37, hook assembly 3660 comprises an elongate hook member 3662 and a pair of opposing jaw members 3664A and 3664B. Jaw members 3664A and 3664B are operably connected to hook member 3662 by spring 3668. In use, a distal end of hook assembly 3660 is positioned adjacent a stent framework and hook member 3662 is moved in the direction of arrow A to move jaw members 3664A and 3664B apart. Those with skill in the art will appreciate that hook member 3662 may be configured so that either jaw member 3664A, 3664B or both may be moved to allow the hook assembly to grasp and retain a portion of the stent. Movement of hook member 3662 in the direction of arrow B closes the jaw members to retain the stent.

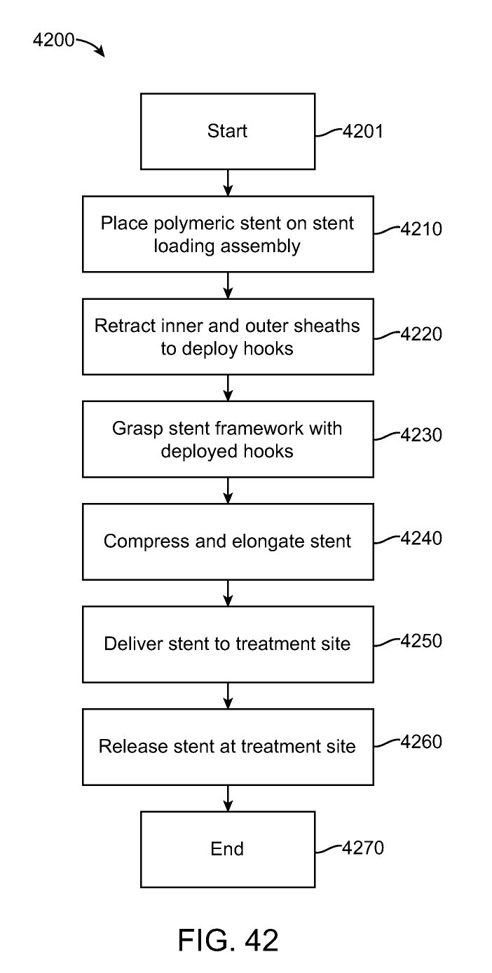

FIG. 42 is a flow chart of one embodiment of a method 4200 of loading and delivering a braided polymeric stent, in accordance with the present invention. Method 4200 begins at 4201.

A braided stent is disposed on a stent loading assembly adjacent a distal end of a delivery catheter (Block 4210). In one embodiment, braided stent is a self-expanding polymeric stent such as stent 120. Stent 120 may be coated with a therapeutic agent. In one embodiment, stent loading assembly comprises stent loading assembly 110. Stent 120 is placed on stent loading assembly in an expanded state. The stent loading and delivery system is processed and stored with the stent in the expanded state. For implantation, the expanded stent is moved from the storage configuration to a delivery configuration just prior to insertion into the patient. To move the stent into the delivery configuration, inner and outer sheaths of the stent loading assembly are retracted to deploy the distal and proximal hooks (Block 4220). The stent framework is grasped by the distal and proximal hooks as the distal and proximal hooks are deployed (Block 4230). Stent 110 is elongated and compressed by translating the inner and outer sheaths (Block 4240). The compressed stent is delivered to the treatment site (Block 4250). Delivery to the treatment site may take any path suitable for the particular application. In one embodiment, the delivery catheter is retracted to expose the stent at the treatment site within the targeted lesion prior to release of the stent from the delivery catheter. At the treatment site, the delivered stent is released from the delivery catheter (Block 4260). In one embodiment, the stent is released when the distal and proximal hooks are covered by the inner and outer sheaths. In one embodiment, the hooks may be covered by the respective sheaths by moving the sheaths in a distal direction while maintaining the hook members substantially stationary. Alternatively, the hook members may be retracted in a proximal direction into their respective sheaths. After the hooks are covered the delivery catheter can be removed from the patient. Method 4200 ends at 4270.

It is important to note that FIGS. 1-42 illustrate specific applications and embodiments of the present invention, and are not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

One such modification is that the number and placement of the hook members and/or assemblies may vary from the specific illustrations. Some embodiments may have as few as one hook member and associated hook or as many as ten hook members and associated hooks configured to grasp and retain various portions of the stent framework.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent loading and delivery system, the system comprising:
a delivery catheter having a catheter lumen;
a stent loading assembly adjacent a distal end of the delivery catheter; and
a stent disposed on the stent loading assembly, wherein the stent comprises a braided stent framework having a first framework end and a second framework end.

2. The stent loading and delivery system of claim 1 wherein the stent loading assembly comprises:
an elongate inner sheath having a lumen;
at least one elongate distal hook member disposed within the inner sheath lumen;
an elongate outer sheath having a lumen; and
at least one elongate proximal hook member disposed within the outer sheath lumen, the proximal hook members disposed between an inner surface of the outer sheath and an outer surface of the inner sheath.

3. The stent loading and delivery system of claim 2 wherein the at least one distal hook member includes a hook at a distal end of the distal hook member and the at least one proximal hook member includes a hook at a distal end of the proximal hook member.

4. The stent loading and delivery system of claim 3 wherein the at least one distal hook and at least one proximal hook are configured to grasp the braided stent framework.

5. The stent loading and delivery system of claim 2 further comprising a balloon disposed on an outer surface of the elongate inner sheath

6. The stent loading and delivery system of claim 1 wherein the delivery catheter comprises a retractable sheath.

7. The stent loading and delivery system of claim 6 further comprising a balloon disposed on an outer surface of the elongate inner sheath.

8. The stent loading and delivery system of claim 1 wherein the stent loading assembly comprises:
a pushrod having a handle and an elongated rod portion, the elongated rod portion extending from the handle; and
an elongated cinch, the cinch having a first end and a second end, wherein the cinch is removably disposed around the first framework end, the cinch configured to compress the first framework end about the elongated rod portion.

9. The stent loading and delivery system of claim 1 wherein the stent loading assembly comprises:
an elongated inner member and a collapsible stopper releasably attached to a distal end of the inner member, the collapsible stopper abutting a distal end of the stent, and
wherein the delivery catheter includes a funnel removably attached to a distal end of the delivery catheter.

10. The stent loading and delivery system of claim 9 wherein the funnel is attached to the distal end of the delivery catheter at a perforated junction.

11. The stent loading and delivery system of claim 9 wherein the funnel is attached to the distal end of the delivery catheter using adhesive.

12. The stent loading and delivery system of claim 1 wherein the stent loading assembly comprises:
an elongated inner member disposed within the catheter lumen; and
an elongated cinch, the cinch having a first end and a second end, wherein the cinch is removably disposed around the second framework end, the cinch configured to compress the second framework end about the elongated inner member.

13. The stent loading and delivery system of claim 1 further comprising:
a funnel, the funnel detachably connected to a distal end of the delivery catheter, and
an inner catheter disposed with the catheter lumen of the delivery catheter, the inner catheter having an inner catheter lumen, the inner catheter lumen having a first diameter,
wherein the stent loading assembly comprises a collapsible stopper and an inner member extending from the collapsible stopper, the inner member having a second diameter, the second diameter less than the first diameter of the inner catheter lumen.

14. The stent loading and delivery system of claim 1 further comprising:
a funnel, the funnel detachably connected to a distal end of the delivery catheter, and wherein the stent loading assembly comprises:
at least one elongate hook member, the at least one hook member having a hook disposed at a distal end of the hook member, the hook configured to grasp and release the stent framework.

15. The stent loading and delivery system of claim 1 wherein the braided stent framework is composed of a material chosen from the group consisting of bioabsorbable material, biodegradable material, non-degradable material and combinations thereof.

16. The stent loading and delivery system of claim 15 wherein the bioabsorbable material is a homopolymer or copolymer of a group of monomers, the group of monomers consisting of: glycolide, p-dioxanone, lactide, ε-caprolactone, or trimethylene carbonate (TMC), or any blend or ratio combination thereof.

17. The stent loading and delivery system of claim 1 wherein the stent loading assembly includes at least one mechanical hook, the mechanical hook configured to grasp and release the stent framework.

18. A method of loading and delivering a braided stent, the method comprising:
providing a braided stent, the braided stent disposed on a stent loading assembly;
retracting an inner sheath and an outer sheath;
deploying at least one hook from the inner sheath and at least one hook from the outer sheath based on the retraction;
grasping a portion of the braided stent with each of the deployed hooks; and
elongating and compressing the grasped stent.

19. The method of claim 18 further comprising:
delivering the compressed stent to a treatment site; and
releasing the stent at the treatment site.

20. The method of claim 19 wherein releasing the stent at the treatment site comprises retracting an outer sheath.

21. A method of loading and delivering a braided stent, the method comprising:
providing a braided stent, the braided stent disposed on a stent loading assembly;
inserting the stent loading assembly into a delivery catheter;
compressing the braided stent based on the insertion of the stent loading assembly into the delivery catheter; and
loading the stent within the delivery catheter based on the compression of the braided stent.

22. The method of claim 21 wherein compressing the braided stent comprises contacting the braided stent with an inner surface of a funnel, the funnel detachable connected to a distal end of the delivery catheter.

23. The method of claim 21 further comprising:
cinching an end of the braided stent to a portion of the stent loading assembly prior to inserting the stent loading assembly into a delivery catheter.
